(19) United States
(12) Patent Application Publication
(10) Pub. No.: US 2023/0124631 A1
(43) Pub. Date: Apr. 20, 2023

(54) HAIR STYLING COMPOSITION I CONTAINING SUGAR

(71) Applicant: Henkel AG & Co. KGaA, Duesseldorf (DE)

(72) Inventors: Thorsten KNAPPE, Schenefeld (DE); Anja THAMMASIRI, Klein Nordende (DE)

(73) Assignee: Henkel AG & Co. KGaA, Duesseldorf (DE)

(21) Appl. No.: 17/784,549

(22) PCT Filed: Oct. 13, 2020
(86) PCT No.: PCT/EP2020/078718

§ 371 Date: Jun. 10, 2022

(30) Foreign Application Priority Data
Dec. 12, 2019 (DE) 102019219410.9

(51) Int. Cl. A61K 8/60; A61K 8/86; A61Q 5/06; A61K 8/33

(52) U.S. Cl. CPC A61K 8/60; A61K 8/86; A61Q 5/06; A61K 8/33

(57) ABSTRACT

The present disclosure concerns a styling composition comprising at least one disaccharide and at least one propellant, wherein the composition is free of synthetic film-forming polymers.

### CROSS-REFERENCE TO RELATED APPLICATION

This application is a U.S. National-Stage entry under 35 U.S.C. § 371 based on International Application No. PCT/EP2020/078718, filed Oct. 13, 2020, which was published under PCT Article 21(2) and which claims priority to German Application No. 102019219410.9, filed Dec. 12, 2019, which are all hereby incorporated in their entirety by reference.

### TECHNICAL FIELD

The present disclosure concerns sugar hair styling compositions and their use for temporarily reshaping keratinous fibers.

Styling compositions for temporarily reshaping keratinous fibers, preferably hair, are known as styling products and are commonly used in the form of gels, pomades, waxes, foams and sprays.

### BACKGROUND

Keratinous fibers include all fibers comprising keratin, such as wool, furs and feathers, in particular human hair, especially preferably human head hair. In a temporary reshaping of keratinous fibers, no chemical (oxidation and reduction) process takes place, rather the fibers are shaped by the application of styling products comprising strengthening agents, such as waxes and/or polymers.

To fix hairstyles while not negatively affecting the shine and healthy and natural look of the hair, common styling products often contain synthetic polymers. These are often produced from synthetic monomers obtained by refining fossil raw materials, such as crude oil, with increased energy consumption. The synthetically produced polymers are often hardly (biologically) degradable.

As part of a more conscious approach to nature and the finite nature of raw material resources, there is an increasing demand from final consumers for natural, sustainable products, i.e. products produced using regenerative raw materials, which have been created using as little energy as possible.

Therefore, the aim of current research is to find natural components that, when used in compositions for the temporary reshaping of keratinous fibers, give them the same positive properties as the synthetic film-forming polymers used to date. In addition to excellent hold, you should also give the hairstyle shine, elasticity and smoothness and avoid the formation of a visible film on the hair, which makes it look dull. Another requirement for these natural raw materials is their processability into stable and user-friendly compositions.

Therefore, it is the task of the present disclosure to provide a composition for temporary shaping of keratinous fibers, which comprises mainly natural ingredients and offers the same hold, shine and smoothness as known styling products with synthetic components.

### BRIEF SUMMARY

The present disclosure provides a styling composition comprising:
a) at least one disaccharide and
b) at least one blowing agent,
wherein the composition is substantially free of synthetic film-forming polymers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description. It is to be appreciated that all numerical values as provided herein, save for the actual examples, are approximate values with endpoints or particular values intended to be read as “about” or “approximately” the value as recited.

The task is solved by providing a styling composition comprising
a) at least one disaccharide and
b) at least one blowing agent,
wherein the composition is free from synthetic film-forming polymers.

Styling composition means a cosmetic composition, which may be formulated in conventional forms of styling compositions, preferably the styling composition is sprayable, particularly preferably it is a spray formulation.

The preferred cosmetically acceptable carrier component of the styling composition is water, particularly preferably distilled water, most preferably distilled water without H₂O₂. The proportion of water in the styling composition is 10 to 60% by weight, preferably 15 to 50% by weight, particularly preferably 20 to 45% by weight, most preferably 25 to 35% by weight, based on the total proportion of the styling composition

In a particularly preferred embodiment of the present disclosure, the styling composition comprises sufficient water to complete the remaining components included in the styling composition to 100% by weight, based on the total weight of the styling composition, also referred to as “ad. 100”.

The sum of all components of the styling composition is 100% by weight, based on the total amount of the styling composition.

For the purposes of the present disclosure, disaccharide refers to two-sugars composed of two monosaccharides selected from glucose, fructose, galactose, rhamnose and xylose. Preferred disaccharides are selected from sucrose, lactose, lactulose, trehalose, maltose, maltulose, cellobiose, gentobiose, isomaltose, isomaltulose, laminaribiose, melibiose, neohesperidose, neotrehalose, nigerose, rutinose, sambubiose, sophorose and mixtures thereof, particularly preferred from sucrose, lactose, maltose, trehalose and mixtures thereof.

Preferably, the at least one disaccharide comprises sucrose (INCI: Sucrose). In a preferred embodiment, the at least one disaccharide is sucrose (INCI: Sucrose). Preferably, the at least one blowing agent (blowing gas) is selected from branched or unbranched C₁-C₅ hydrocarbons, which may optionally be halogenated, dimethyl ether, N₂O, air and mixtures thereof.

Propellants as contemplated herein are all gases approved for cosmetic compositions, in particular the propellants are selected from propane, propene, n-butane, iso-butane, iso-butene, 2-methylbutane, n-pentane, Pentene, iso-pentane, iso-pentene, methane, ethane, dimethyl ether, nitrogen, air, oxygen, nitrous oxide, 1, 1, 1, 3-tetrafluoroethane, heptafluoro-n-propane, perfluoroethane, monochlorodifluoromethane, 1-difluoroethane (INCI: Hydrofluorocarbon 152a) and mixtures thereof, particularly preferred are diethyl ether, propane, n-butane, iso-butane as well as mixtures thereof, even more preferred is diethyl ether.

Preferably, the at least one disaccharide is present in the styling composition at a level of 1.0 to 20.0% by weight, preferably 4.0 to 17.0% by weight, more preferably 5.0 to 13.0% by weight, even more preferably 6.0 to 10.0% by weight, based on the total amount of the styling composition.

Preferably, the at least one blowing agent is present in the styling composition at a level of 10.0 to 65.0% by weight, preferably 15.0 to 55.0% by weight, more preferably 25.0 to 45.0% by weight, even more preferably 30.0 to 40.0% by weight, based on the total amount of the styling composition.

The styling composition is free of synthetic, film-forming polymers. Synthetic film-forming polymers are all polymers, which leave an at least partially continuous film when drying, especially on the hair. Furthermore, film-forming polymers are those polymers which, when used in 0.01 to 20% by weight, preferably 1 to 5% by weight, aqueous, alcoholic or aqueous-alcoholic solution, are capable of crosslinking and/or stiffening hair, for example of a strand of hair, by film formation. Preferably, crosslinking or stiffening is achieved by depositing a transparent polymer film on the hair.

Preferably, the styling composition does not contain a synthetic film-forming polymer, in particular a homo- or copolymer, comprising monomer units selected from siloxane, vinylpyrrolidone, vinylcaprolactam, N-vinylformamide, vinyl ester, such as vinyl acetate, Vinyl alcohol, acrylamide, methacrylamide, C1 to C7 alkyl acrylamide, C1 to C7 dialkyl acrylamide, C1 to C7 alkyl methacrylamide, C1 to C7 dialkyl methacrylamide, C1 to C7 alkyl acrylate, acrylic acid, propylene glycol, maleic acid, styrene, itaconic acid and mixtures thereof.

Particularly preferably, the styling composition is free of synthetic film-forming polymers selected from vinylpyrrilidone/vinyl ester copolymers, polyvinylpyrrilidones, siloxanes, glycosidically substituted silicones, homopolyacrylic acid and polyvinylpyrrolidones, in particular acrylamides/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamides Copolymer, Aminoethylpropanediol-AMPD-Acrylates/Diacetoneacrylamides Copolymer, Ammonium VA/Acrylates Copolymer, AMPD-Acrylates/Diacetoneacrylamides Copolymer, AMP-Acrylates/Allyl Methacrylates Copolymer, AMP Acrylates/C1-18 Alkyl Acrylates/C1-8 Alkyl Acrylamides Copolymer, AMP Acrylates/Diacetoneacrylamide Copolymer, AMP Acrylates/Dimethylaminoethyl Methacrylates Copolymer, Bis-Butyloxyamodimethicone/PEG-60 Copolymer, Butyl Acrylates/Ethylhexyl Methacrylates Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Diethylene Glycolamine/Epichlorohydrin/Piperazine Copolymer, Dimethicone Crosspolymer, Diphenyl Amodimethicone, Ethyl ester of PVM/MA copolymer, Isobutylene/ethylmaleimide/hydroxyethylmaleimide copolymer, Isobutylene/MA copolymer, Isobutyl methacrylate/bis-hydroxypropyl dimethicone acrylate copolymer, Isopropyl ester of PVM/MA copolymer, Lauryl acrylate crosspolymer, Lauryl Methacrylate/Glycol Dimethacrylate Crosspolymer, MEA sulfites, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 dimethicone/acrylates copolymer, PEG-8/SMDI copolymer, polyacrylamide, polyacrylate-6, polybutylene terephthalate, polyester-1, polyethylacrylate, polyethylene terephthalate, polymethacryloyl ethyl betaine, polypentaerythrityl terephthalate, Polyperfluoroperhydrophenanthrene, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Sodium Butyl Ester of PVM/MA Copolymer, Sodium Ethyl Ester of PVM/MA Copolymer, Sodium Polyacrylate, Terephthalic Acid/Isophthalic Acid/Sodium Isophthalic Acid Sulfonate/Glycol Copolymer, Trimethylolpropane Triacrylate, Trimethyl siloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/acrylates/lauryl methacrylate copolymer, VP/dimethylaminoethyl methacrylate copolymer, VP/DMAPA acrylates copolymer, VP/hexadecene copolymer, VP/VA copolymer, VP/vinyl caprolactam/DMAPA acrylates copolymer, and styrene/VP copolymer polyimides, such as polyimide-1.

Substantially free of synthetic, film-forming polymers, as referred to herein, means that the proportion of synthetic, film-forming polymers in the styling composition is a maximum of 0.5% by weight, preferably a maximum of 0.25% by weight, particularly preferably a maximum of 0.1% by weight, even more preferably a maximum of 0.05% by weight, even more preferably 0.0% by weight, based on the total proportion of the styling composition. In some embodiments, no synthetic, film-forming polymers are present at all.

In one embodiment as contemplated herein, the styling composition comprises polyethylene glycols (PEG), which can be described by the general formula H—(O—CH2—CH2)n—OH. PEGs exhibit a degree of polymerization n of about 5 up to <100,000, corresponding to molar masses of 200 to 5,000,000 gmol-1. The products with molecular masses below 25,000 gmol-1 are referred to as polyethylene glycols proper, while higher molecular mass products are often referred to in the literature as polyethylene oxides (abbr: PEOX) can be designated.

The polyethylene glycols preferably used may have a linear or branched structure, with linear polyethylene glycols being particularly preferred.

Preferably, the styling composition further comprises at least one polyethylene glycol having 3 to 20 ethylene oxide units, preferably having 4 to 15, more preferably having 5 to 13, most preferably having 6 to 10, even more preferably 8 ethylene oxide units.

Suitable water-soluble polyethylene glycols are consequently selected from PEG-3, PEG-4, PEG-6, PEG-7, PEG-8, PEG-9, PEG-10, PEG-12, PEG-14, PEG-16, PEG-18, PEG-20 and mixtures thereof, preferably PEG-4 to PEG-12, particularly preferred is PEG-8.

The preferred polyethylene glycols have relative molecular masses in the range of 100 and 2000, preferably from 200 to 1500, even more preferably from 200 to 1000, most preferred are PEGs, which are in the liquid state at room temperature and a pressure of 1 bar, preferably polyethylene glycol with a relative molecular mass of 200, 400 and 600.

Preferably, the styling composition further comprises at least one alcohol, preferably at least one aliphatic, branched or unbranched, monohydric alcohol with 1 to 9, preferably with 2 to 6, particularly preferably with 2 to 4 C atoms. Preferably, the alcohol is selected from the group consisting of methanol, ethanol, n-propanol, isopropanol, n-butanol, tert-butanol, n-pentanol, iso-pentanol, n-hexanol, iso-hexanol and isobutanol. Ethanol is the most preferred.

In a preferred embodiment of the present disclosure, the styling composition comprises two different alcohols selected from ethanol, n-propanol, isopropanol, n-butanol, tert-butanol, particularly preferably the styling composition comprises ethanol and tert-butanol.

In a particular embodiment of the present disclosure, the styling composition further comprises at least one polyol preferably having from 2 to 15 carbon atoms and at least two hydroxy groups, more preferably the polyol has from 2 to 9 carbon atoms and from 2 to 6 hydroxy groups.

Particularly preferably, the at least one polyol is selected from glycerol, alkylene glycerol, such as ethylene glycol, diethylene glycol, propylene glycol, such as 1,2-propylene glycol, 1,3-propylene glycol, 2-methyl-1,3-propanediol, butylene glycol, such as 1,2-butylene glycol, 1,3-butylene glycol and 1,4-butylene glycol, pentylene glycol, such as 1,2-pentanediol and 1,5-pentanediol, hexylene glycol, such as 1,6-hexanediol, Hexanetriols such as 1,2,6-hexanetriol, 1,2-octanediol, 1,8-octanediol and methylol compounds such as trimethylolethane, trimethylolpropane and trimethylolbutane, dipropylene glycol, tripropylene glycol, Diglycerol, triglycerol, erythritol, pentaerythritol, dipentaerythritol, trimethylolpropane, sorbitol, monoanhydrosorbites (sorbitans), and cyclohexanetriol, most preferably the polyol glycerol.

Even more particularly preferred, the polyol is water soluble.

Preferably, the at least one polyol is present in the styling composition in an amount of from 0.05% to 5.0% by weight, more preferably from 0.1% to 3.0% by weight, most preferably from 0.3% to 2.0% by weight, even more preferably from 0.5% to 1.0% by weight.

In another embodiment, the styling composition may comprise the at least one alcohol, preferably ethanol, and the at least one polyol, preferably glycerol. In a still more preferred embodiment, the styling composition comprises two of the alcohols, preferably ethanol and tert-butanol, and the at least one polyol, preferably glycerol.

Preferably, the at least one polyethylene glycol is included in the styling composition at a level of 0.1 to 8.0% by weight, preferably 0.5 to 5.5% by weight, more preferably 1.0 to 4.0% by weight, even more preferably 1.5 to 3.0% by weight, based on the total amount of the styling composition.

Preferably, the at least one alcohol is present in the styling composition at a level of 5.0 to 55.0% by weight, preferably 10.0 to 45.0% by weight, more preferably 15.0 to 35.0% by weight, even more preferably 20.0 to 30.0% by weight, based on the total amount of the styling composition.

In a particular embodiment of the present disclosure, the styling composition may additionally comprise at least one nonionic, anionic, cationic and/or amphoteric or zwitterionic surfactant. Examples of anionic surfactants include soaps, alkylbenzene sulfonates, alkane sulfonates, olefin sulfonates, alkyl ether sulfonates, glycerol ether sulfonates, α-methyl ester sulfonates, sulfo fatty acids, Alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, Mono- and dialkylsulfosuccinamates, sulfotriglyceride, amide soaps, ether carboxylic acids and their salts, fatty acid isethionates, fatty acid sarcosinates, fatty acid taurides, N-acyl amino acids, such as acyl lactylates, acyl tartrates, acyl glutamates and acyl partates, alkyl oligoglucoside sulfates, protein fatty acid condensates (especially wheat-based vegetable products) and alkyl (ether) phosphates. If the anionic surfactants contain polyglycol ether chains, these may have a conventional, but preferably a narrowed homolog distribution. Examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or Mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. Where the nonionic surfactants contain polyglycol ether chains, these may have a conventional, but preferably a narrowed homolog distribution. Examples of cationic surfactants are quaternary ammonium compounds and esterquats, in particular quaternized fatty acid trialkanolamine ester salts. Typical examples of amphoteric or zwitterionic surfactants are alkyl betaines, alkylamidobetaines, aminopropionates, aminoglycinates, imidazolinium betaines and sulfobetaines. The surfactants mentioned are exclusively known compounds. With regard to the structure and production of these substances, reference should be made to relevant reviews, for example J.Falbe (ed.), “Surfactants in Consumer Products”, Springer Verlag, Berlin, 1987, pp. 54-124 or J.Falbe (ed.), “Katalysatoren, Tenside and Mineraloladditive”, Thieme Verlag, Stuttgart, 1978, pp. 123-217.

Preferred surfactants or emulsifiers or also surface-active substances are PEG derivatives of hydrogenated castor oil, which are available, for example, under the designation PEG Hydrogenated Castor Oil, e.g. PEG-30 Hydrogenated Castor Oil, PEG-33 Hydrogenated Castor Oil, PEG-35 Hydrogenated Castor Oil, PEG-36 Hydrogenated Castor Oil, PEG-40 Hydrogenated Castor Oil or PEG-60 Hydrogenated Castor Oil. Preferred is PEG-60 Hydrogenated Castor Oil. The proportion of the at least one surfactant in the styling composition is preferably 0.05 to 3.5% by weight, particularly preferably 0.1 to 2.5% by weight, most preferably 0.2 to 1.0% by weight, based on the total proportion of the styling composition.

Furthermore, the styling composition may contain pH adjusting agents. Preferred agents are, for example, primary amino alcohols such as aminomethyl propanol (INCI), which is commercially available under the name AMP-ULTRA© PC, for example AMP-ULTRA© PC 2000. Further agents for adjusting the pH can be selected from alkali hydroxides, in particular sodium hydroxide and potassium hydroxide, organic acids, such as lactic acid, citric acid, hydrochloric acid, sulfuric acid, phosphoric acid, adipic acid, hydroxysuccinic acid, succinic acid, tartaric acid or malic acid, in particular from tetrahydroxypropyl ethylenediamine, known under the trade name Neutrol TE, and triethylamine (TEA).

The proportion of the pH-adjusting agents in the styling composition is preferably 0.01 to 1.0% by weight, particularly preferably 0.05 to 0.1% by weight, most preferably 0.05 to 0.08% by weight, based on the total proportion of the styling composition.

Furthermore, the styling composition may contain a preservative, in particular those listed in the Cosmetic Products Regulation in Annex V (Regulation (EC) No 122+3/2009 of the European Parliament and of the Council of Nov. 30, 2009 on cosmetic products), especially preferably benzoic acid, benzoates and mixtures thereof, especially preferably sodium benzoate.

The proportion of preservatives in the styling composition is preferably 0.05 to 1.0% by weight, particularly preferably 0.075 to 0.6% by weight, most preferably 0.1 to 0.4% by weight, based on the total proportion of the styling composition.

Preferably, the styling composition is used for temporarily reshaping keratinous fibers, in particular human scalp hair. Furthermore, it is preferably used for texturizing keratinous fibers, especially human scalp hair. Texturing, as contemplated herein, refers to changing the quality of the hair surface of a hairstyle.

It has been shown that the combination of components as contemplated herein results in a styling composition that meets the current increasing consumer demand for products with natural ingredients and, at the same time, is particularly well suited for temporarily reshaping keratinous fibers, preferably human scalp hair, while keeping the hairstyle in the desired shape, at the same time retaining its lightness and appearing neither weighed down nor stiff.

Analyzed Example

The composition of some other preferred styling compositions can be found in the following tables (data in % by weight based on the total weight of the styling composition unless otherwise stated).

| Component | Formula 1 | Formula 2 | Formula 3 | Formula 4 | Formula 5 |
|---|---|---|---|---|---|
| Disaccharide | 1.0-20.0 | 5.0-17.0 | 6.0-13.0 | 6.0-10.0 | 6.5-9.0 |
| Propellant | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| optional components and | ad. 100 | ad. 100 | ad. 100 | ad. 100 | ad. 100 |
| water | | | | | |

| Component | Formula 6 | Formula 7 | Formula 8 | Formula 9 | Formula 10 |
|---|---|---|---|---|---|
| Sucrose | 1.0-20.0 | 5.0-17.0 | 6.0-13.0 | 6.0-10.0 | 6.5-9.0 |
| Propellant | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| optional components and | ad. 100 | ad. 100 | ad. 100 | ad. 100 | ad. 100 |
| water | | | | | |
| Component | Formula 11 | Formula 12 | Formula 13 | Formula 14 | Formula 15 |
| Disaccharide | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| optional components and | ad. 100 | ad. 100 | ad. 100 | ad. 100 | ad. 100 |
| water | | | | | |
| Component | Formula 16 | Formula 17 | Formula 18 | Formula 19 | Formula 20 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| optional components and | ad. 100 | ad. 100 | ad. 100 | ad. 100 | ad. 100 |
| water | | | | | |
| Component | Formula 21 | Formula 22 | Formula 23 | Formula 24 | Formula 25 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| Polyethylene glycol with | 0.1-8.0 | 0.5-5.5 | 1.0-4.0 | 1.0-3.0 | 1.2-2.0 |
| 2-20 EO units | | | | | |
| optional components and | ad. 100 | ad. 100 | ad. 100 | ad. 100 | ad. 100 |
| water | | | | | |
| Component | Formula 26 | Formula 27 | Formula 28 | Formula 29 | Formula 30 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| optional components and | ad. 100 | ad. 100 | ad. 100 | ad. 100 | ad. 100 |
| water | | | | | |
| Component | Formula 31 | Formula 32 | Formula 33 | Formula 34 | Formula 35 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| monohydric alcohol with | 5.0-55.0 | 10.0-45.0 | 15.0-35.0 | 20.0-30.0 | 20.0-25.0 |
| C2 to C6 | | | | | |
| optional components and | ad. 100 | ad. 100 | ad. 100 | ad. 100 | ad. 100 |
| water | | | | | |
| Component | Formula 36 | Formula 37 | Formula 38 | Formula 39 | Formula 40 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| Ethanol | 5.0-55.0 | 10.0-45.0 | 15.0-35.0 | 20.0-30.0 | 20.0-25.0 |
| optional components and | ad. 100 | ad. 100 | ad. 100 | ad. 100 | ad. 100 |
| water | | | | | |
| Component | Formula 41 | Formula 42 | Formula 43 | Formula 44 | Formula 45 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| Ethanol | 5.0-55.0 | 10.0-45.0 | 15.0-35.0 | 20.0-30.0 | 20.0-25.0 |
| Polyol with 2-5 C atoms | 0.5-5.0 | 0.1-3.0 | 0.3-2.0 | 0.5-1.0 | 0.6-0.9 |
| optional components and | ad. 100 | ad. 100 | ad. 100 | ad. 100 | ad. 100 |
| water | | | | | |
| Component | Formula 46 | Formula 47 | Formula 48 | Formula 49 | Formula 50 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| Ethanol | 5.0-55.0 | 10.0-45.0 | 15.0-35.0 | 20.0-30.0 | 20.0-25.0 |
| Glycerin | 0.5-5.0 | 0.1-3.0 | 0.3-2.0 | 0.5-1.0 | 0.6-0.9 |
| optional components and | ad. 100 | ad. 100 | ad. 100 | ad. 100 | ad. 100 |
| water | | | | | |
| Component | Formula 51 | Formula 52 | Formula 53 | Formula 54 | Formula 55 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| Ethanol | 5.0-55.0 | 10.0-45.0 | 15.0-35.0 | 20.0-30.0 | 20.0-25.0 |
| Glycerin | 0.5-5.0 | 0.1-3.0 | 0.3-2.0 | 0.5-1.0 | 0.6-0.9 |
| Surfactant | 0.05-3.5 | 0.1-2.5 | 0.2-2.0 | 0.2-1.0 | 0.2-0.5 |
| optional components and | ad. 100 | ad. 100 | ad. 100 | ad. 100 | ad. 100 |
| water | | | | | |
| Component | Formula 56 | Formula 57 | Formula 58 | Formula 59 | Formula 60 |
| Sucrose | 1.0-20.0 | 5.0-17.0 | 6.0-13.0 | 6.0-10.0 | 6.5-9.0 |
| Diethyl ether | 10.0-65.0 | 15.0-55.0 | 25.0-45.0 | 30.0-40.0 | 33.0-38.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.2-2.0 |
| Ethanol | 5.0-55.0 | 10.0-45.0 | 15.0-35.0 | 20.0-30.0 | 20.0-25.0 |
| Glycerin | 0.5-5.0 | 0.1-3.0 | 0.3-2.0 | 0.5-1.0 | 0.6-0.9 |
| nonionic surfactant | 0.05-3.5 | 0.1-2.5 | 0.2-2.0 | 0.2-1.0 | 0.2-0.5 |
| optional components and | ad. 100 | ad. 100 | ad. 100 | ad. 100 | ad. 100 |
| water | | | | | |
| Component | Formula 61 | Formula 62 | Formula 63 | Formula 64 | Formula 65 |
| Sucrose | 1.0-20.0 | 5.0-17.0 | 6.0-13.0 | 6.0-10.0 | 8.0 |
| Dimethyl ether | 10.0-65.0 | 15.0-55.0 | 25.0-45.0 | 30.0-40.0 | 35.0 |
| synthetic, film-forming | 0.0 | 0.0 | 0.0 | 0.0 | 0.0 |
| polymers | | | | | |
| PEG-8 | 0.1-8.0 | 0.5-5.5 | 1.0-4.0 | 1.0-3.0 | 1.5 |
| Ethanol denat. | 5.0-55.0 | 10.0-45.0 | 15.0-35.0 | 20.0-30.0 | 23.0 |
| tert-butanol | 0.001-0.1 | 0.005-0.08 | 0.009-0.05 | 0.01-0.03 | 0.02 |
| Glycerin | 0.5-5.0 | 0.1-3.0 | 0.3-2.0 | 0.5-1.0 | 0.7 |
| Hydrogenated Castor Oil | 0.05-3.5 | 0.1-2.5 | 0.2-2.0 | 0.2-1.0 | 0.4 |
| 60EO | | | | | |
| Water distilled, without | ad. 100 | ad. 100 | ad. 100 | ad. 100 | ad. 100 |
| H₂O₂ | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A styling composition comprising:
a) at least one disaccharide and
b) at least one blowing agent,
wherein the composition is substantially free of synthetic film-forming polymers.

2. The styling composition of claim 1, wherein the at least one disaccharide comprises sucrose.

3. A styling composition according to claim 1, wherein the at least one blowing agent is selected from branched or unbranched optionally halogenated C₁-C₅ hydrocarbons, dimethyl ether, N₂O, CO₂, -air and mixtures thereof.

4. A styling composition according to claim 1, wherein the at least one disaccharide is present in the styling composition in a proportion of 1.0 to 20.0% by weight, based on the total proportion of the styling composition.

5. A styling composition according to claim 1, wherein the at least one blowing agent is present in the styling composition in a proportion of 10.0 to 65.0% by weight, based on the total proportion of the styling composition.

6. A styling composition according to claim 1, wherein the styling composition is free from a synthetic film-forming polymer.

7. A styling composition according to claim 1, wherein the styling composition further comprises at least one polyethylene glycol having from 3 to 20 ethylene oxide units.

8. A styling composition according to claim 1, further comprising at least one alcohol.

9. A styling composition according to claim 7, wherein the at least one polyethylene glycol is present in the styling composition in a proportion of 0.1 to 8.0% by weight, based on the total proportion of the styling composition.

10. A styling composition according to claim 8, wherein the at least one alcohol is present in the styling composition in a proportion of 5.0 to 55.0% by weight, based on the total proportion of the styling composition.

11. (canceled)

12. A styling composition of claim 1, wherein the at least one disaccharide consists of sucrose.

13. A styling composition according to claim 1, wherein the at least one disaccharide is present in the styling composition in a proportion of 6.0 to 10.0% by weight, based on the total proportion of the styling composition.

14. A styling composition according to claim 13, wherein the at least one blowing agent is present in the styling composition in a proportion of 30.0 to 40.0% by weight, based on the total proportion of the styling composition.

15. A styling composition according to claim 1, wherein the styling composition is free from a homo- or copolymer comprising monomer units selected from siloxane, vinylpyrrolidone, vinylcaprolactam, N-vinylformamide, vinyl ester, vinyl alcohol, acrylamide, methacrylamide, C1- to C7-alkylacrylamide, C1- to C7-dialkylacrylamide, C1- to C7-alkylmethacrylamide, C1- to C7-dialkylmethacrylamide, C1- to C7-alkylacrylate, acrylic acid, propylene glycol, maleic acid, styrene, itaconic acid and mixtures thereof.

16. A styling composition according to claim 1, wherein the styling composition further comprises at least one polyethylene glycol having from 6 to 10 ethylene oxide units.

17. A styling composition according to claim 16, further comprising at least one aliphatic, branched or unbranched, monohydric alcohol having 1 to 9 C atoms.

18. A styling composition according to claim 16, further comprising at least one alcohol selected from the group consisting of ethanol, n-propanol, isopropanol, n-butanol, tert-butanol and isobutanol.

19. A styling composition according to claim 14, further comprising at least one polyethylene glycol having from 3 to 20 ethylene oxide units and present in the styling composition in a proportion of 1.5 to 3.0% by weight, based on the total proportion of the styling composition, and at least one alcohol is present in the styling composition in a proportion of 20.0 to 30.0% by weight, based on the total proportion of the styling composition.
